(19) United States
(12) Patent Application Publication
(10) Pub. No.: US 2017/0113982 A1
(43) Pub. Date: Apr. 27, 2017

(54) PROCESS FOR THE OXIDATIVE DEHYDROGENATION OF ETHANE

(71) Applicant: SHELL OIL COMPANY, Houston, TX (US)

(72) Inventors: Georgios MITKIDIS, Amsterdam (NL); Maria SAN ROMAN MACIA, Amsterdam (NL); Guus ROSSUM VAN, Amsterdam (NL)

(21) Appl. No.: 15/297,428

(22) Filed: Oct. 19, 2016

(30) Foreign Application Priority Data
Oct. 21, 2015 (EP) 15190869.6

(51) Int. Cl. C07C 5/48

(52) U.S. Cl. CPC C07C 5/48

(57) ABSTRACT

The invention relates to a process for the oxidative dehydrogenation of ethane to ethylene which comprises subjecting a gas stream comprising one or more light components, ethane and ethylene, resulting from subjecting ethane and oxygen to ethane oxidative dehydrogenation conditions, to two consecutive sorption steps which comprise contacting said gas stream comprising one or more light components, ethane and ethylene with a selective sorption agent, resulting in sorption of part of said gas stream, comprising ethylene and ethane, by the sorption agent and in a gas stream comprising the remainder of said gas stream, comprising one or more light components and ethane, which latter gas stream is subjected to a second sorption step resulting in a further separation, finally resulting in a gas stream comprising ethane which is optionally recycled to the ethane oxidative dehydrogenation (reaction) step, wherein each sorption step is followed by a desorption step which comprises desorbing the sorbed components originating from the gas stream in question.

### FIELD OF THE INVENTION

The present invention relates to a process for the oxidative dehydrogenation of ethane.

### BACKGROUND OF THE INVENTION

It is known to oxidatively dehydrogenate ethane resulting in ethylene, in an oxidative dehydrogenation (oxydehydrogenation; ODH) process. Examples of ethane ODH processes are for example disclosed in U.S. Pat. No. 7,091,377, WO2003064035, US20040147393, WO2010096909 and US20100256432. The oxidative dehydrogenation of ethane converts ethane into ethylene. In this process, a gas stream comprising ethane is contacted with an ODH catalyst and with an oxidant, such as oxygen or air.

In such ODH process, the oxygen is adsorbed on the catalyst's surface. Ethane molecules are then dehydrogenated into ethylene. Usually, the gas stream leaving an ODH process contains a mixture of water, optionally hydrogen, carbon monoxide, carbon dioxide, optionally methane (coming from the ethane feed), ethane and ethylene. In addition, a certain amount of the corresponding carboxylic acid, that is to say acetic acid, may be formed. That is to say, ethylene is initially formed in an ethane ODH process. However, in said same process, said dehydrogenated compound may be further oxidized under the same conditions into the corresponding carboxylic acid. In the case of ethane, the product of said alkane oxidative dehydrogenation process comprises ethylene and optionally acetic acid.

In general, the yield of ethylene (as determined by conversion and selectivity) that is achieved in an ODH process may be relatively low. As a result, a relatively large amount of unconverted ethane leaves the ODH reactor. The proportion of unconverted ethane in the ODH product gas stream may be up to 80 mole % based on the total molar amount of the gas stream. It is desired to recover and then recycle this unconverted ethane.

It is known to separate ethane from ethylene, by means of cryogenic distillation in so-called “C2 splitter” columns. In such cryogenic distillation, a relatively high pressure and a relatively low (cryogenic) temperature are applied to effect the separation of ethane from ethylene. Generally, such “C2 splitter” is preceded by cryogenic distillation wherein light gases and possibly methane (coming from the ethane feed) are first separated from the ethane and ethylene.

An object of the invention is to provide a technically advantageous, efficient and affordable process for the oxidative dehydrogenation of ethane, including a step wherein a product gas stream comprising (unconverted) ethane and ethylene (product) is separated into a gas stream comprising the ethane and another gas stream comprising the ethylene, more especially in a case where such gas stream to be separated comprises a relatively high proportion of unconverted ethane. Such technically advantageous process would preferably result in a lower energy demand and/or lower capital expenditure.

### SUMMARY OF THE INVENTION

Surprisingly it was found that such technically advantageous process, resulting in a lower energy demand and/or lower capital expenditure, may be provided by subjecting a gas stream comprising one or more light components, ethane and ethylene, resulting from subjecting ethane and oxygen to ethane oxidative dehydrogenation conditions, to two consecutive sorption steps which comprise contacting said gas stream comprising one or more light components, ethane and ethylene with a selective sorption agent, resulting in sorption of part of said gas stream, comprising ethylene and ethane, by the sorption agent and in a gas stream comprising the remainder of said gas stream, comprising one or more light components and ethane, which latter gas stream is subjected to a second sorption step resulting in a further separation, finally resulting in a gas stream comprising ethane which is optionally recycled to the ethane oxidative dehydrogenation (reaction) step, wherein each sorption step is followed by a desorption step which comprises desorbing the sorbed components originating from the gas stream in question.

Accordingly, the present invention relates to a process for the oxidative dehydrogenation of ethane to ethylene, comprising

a reaction step which comprises subjecting a gas stream comprising ethane and oxygen to ethane oxidative dehydrogenation conditions resulting in a gas stream comprising one or more light components, ethane and ethylene;

a first sorption step which comprises contacting the gas stream comprising one or more light components, ethane and ethylene resulting from the reaction step with a sorption agent which has an affinity for light components which is lower than that for ethane which in turn is lower than that for ethylene, resulting in sorption of ethylene and ethane by the sorption agent and in a gas stream comprising one or more light components and ethane;

a first desorption step which comprises desorbing sorbed ethylene and ethane resulting in a gas stream comprising ethylene and ethane;

a second sorption step which comprises contacting the gas stream comprising one or more light components and ethane resulting from the first sorption step with a sorption agent which has an affinity for light components which is lower than that for ethane, resulting in sorption of ethane by the sorption agent and in a gas stream comprising one or more light components;

a second desorption step which comprises desorbing ethane sorbed in the second sorption step resulting in a gas stream comprising ethane; and

optionally a recycle step which comprises recycling the gas stream comprising ethane resulting from the second desorption step to the reaction step.

### BRIEF DESCRIPTION OF THE DRAWINGS

FIG. 1 shows an embodiment of the present invention, in which the gas stream that is subjected to the sorption step additionally comprises components other than ethane, ethylene and one or more light components, namely carbon dioxide.

### DETAILED DESCRIPTION OF THE INVENTION

Within the present specification, by “ethane oxidative dehydrogenation catalyst” reference is made to a catalyst for the oxidative dehydrogenation of ethane. Both terms may be used interchangeably. Analogously, by “ethane oxidative dehydrogenation conditions” reference is made to conditions for the oxidative dehydrogenation of ethane, which terms may also be used interchangeably.

Within the present specification, by “light component” reference is made to a compound of which the boiling point is lower than the boiling point of ethylene. In particular, in the present invention, the light component(s) is or are selected from the group consisting of carbon monoxide (CO), oxygen (O₂), argon (Ar), methane (CH₄), nitrogen (N₂) and hydrogen (H₂). Further in particular, in the present invention, the one or more light components may comprise carbon monoxide, optionally oxygen, optionally argon, optionally methane, optionally nitrogen and optionally hydrogen, but do not comprise carbon dioxide. Said oxygen may be unconverted oxygen originating from the reaction step. Said methane may be methane originating from the ethane feed to the reaction step. Said nitrogen may be nitrogen originating from an air feed to the reaction step.

Within the present specification, by “substantially no” in relation to the amount of a specific component in a gas stream, it is meant an amount which is at most 1,000, preferably at most 500, preferably at most 100, preferably at most 50, more preferably at most 30, more preferably at most 20, and most preferably at most 10 ppmw of the component in question, based on the amount (i.e. weight) of said gas stream.

Within the present specification, where reference is made to relative (e.g. molar) amounts of components in a gas stream, such relative amounts are to be selected such that the total amount of said gas stream does not exceed 100%.

While the process of the present invention and the streams used in said process are described in terms of “comprising”, “containing” or “including” one or more various described steps and components, respectively, they can also “consist essentially of” or “consist of” said one or more various described steps and components, respectively.”.

In the reaction step of the process of the present invention, a gas stream comprising ethane and an oxidant, being either oxygen (O₂) or air (which air comprises oxygen (O₂) and nitrogen (N₂)), is subjected to ethane oxidative dehydrogenation conditions resulting in a gas stream comprising one or more light components, ethane and ethylene. That is to say, in the present invention, oxygen may be fed to the reaction step in the form of air, or in any other form, such as for example the below-described high-purity oxygen.

Thus, in a case wherein in the present invention air is used as the oxidant in the reaction step, nitrogen (originating from such air) is one of the one or more light components in the gas stream resulting from the reaction step. Accordingly, in one embodiment, the present invention concerns a process comprising

a reaction step which comprises subjecting a gas stream comprising ethane and air to ethane oxidative dehydrogenation conditions resulting in a gas stream comprising nitrogen, ethane and ethylene;

a first sorption step which comprises contacting the gas stream comprising nitrogen, ethane and ethylene resulting from the reaction step with a sorption agent which has an affinity for nitrogen which is lower than that for ethane which in turn is lower than that for ethylene, resulting in sorption of ethane and ethylene by the sorption agent and in a gas stream comprising nitrogen and ethane;

a first desorption step which comprises desorbing ethane and ethylene sorbed in the first sorption step resulting in a gas stream comprising ethane and ethylene;

a second sorption step which comprises contacting the gas stream comprising nitrogen and ethane resulting from the first sorption step with a sorption agent which has an affinity for nitrogen which is lower than that for ethane, resulting in sorption of ethane by the sorption agent and in a gas stream comprising nitrogen;

a second desorption step which comprises desorbing ethane sorbed in the second sorption step resulting in a gas stream comprising ethane; and

optionally a recycle step which comprises recycling the gas stream comprising ethane resulting from the second desorption step to the reaction step.

In the above-mentioned reaction step, one gas stream comprising ethane and oxygen or air may be fed to a reactor. Alternatively, two or more gas streams may be fed to the reactor, which gas streams form a combined gas stream comprising ethane and oxygen or air inside the reactor. For example, one gas stream comprising oxygen or air and another gas stream comprising ethane may be fed to the reactor separately.

The reactor may be any reactor suitable for the oxidative dehydrogenation of ethane, such as a fixed bed reactor with axial or radial flow and with inter-stage cooling or a fluidized bed reactor equipped with internal and external heat exchangers. It may be a fixed bed multi-tubular reactor, such as a fixed-bed multi-tube shell-and-tube reactor/heat exchanger with catalyst and process flow inside the tubes and a heat transfer fluid (or steam generation) circulated in the shell side, as for example disclosed in US20100256432.

Preferably, subjecting the gas stream comprising ethane and oxygen or air to ethane oxidative dehydrogenation conditions comprises contacting said gas stream with an ethane oxidative dehydrogenation catalyst, as further described below.

Various processes and reactor set-ups are described in the ODH field and the process of the present invention is not limited in that regard. The person skilled in the art may conveniently employ any of such processes in the reaction step of the process of the present invention.

Suitable oxydehydrogenation processes, including catalysts and other process conditions, include those described in above-mentioned U.S. Pat. No. 7,091,377, WO2003064035, US20040147393, WO2010096909 and US20100256432.

As used herein, the term “reactor feed” is understood to refer to the totality of the gas stream(s) at the inlet(s) of the reactor. Thus, as will be appreciated by one skilled in the art, the reactor feed is often comprised of a combination of one or more gas stream(s), such as an ethane stream, an oxygen stream, an air stream, a recycle gas stream, etc.

During the oxidative dehydrogenation of ethane, a reactor feed comprising ethane and oxygen or air is introduced into the reactor, so that a gas stream comprising ethane and oxygen or air is contacted with an ethane oxidative dehydrogenation catalyst inside that reactor. Optionally, the reactor feed may further comprise minor components of the ethane feed (e.g. methane) or the ethane recycle stream (e.g. ethylene, CO).

In a case wherein in the present invention oxygen (not air) is used as the oxidant in the reaction step, the gas stream comprising oxygen (to be combined with the ethane in the reaction step), may be a high purity oxygen stream. Such high-purity oxygen may have a purity greater than 90%, preferably greater than 95%, more preferably greater than 99%, and most preferably greater than 99.4%.

In the reaction step of the process of the present invention, ethane and oxygen or air may be added to the reactor as mixed feed, optionally comprising further components therein, at the same reactor inlet. Alternatively, the ethane and oxygen or air may be added in separate feeds, optionally comprising further components therein, to the reactor at the same reactor inlet or at separate reactor inlets.

In the reaction step of the process of the present invention, the ethane:oxygen molar ratio (wherein the oxygen may or may not originate from air) in the reactor feed may be in the range of from 0.3:1 to 10:1, more preferably 0.7:1 to 5:1. In a case wherein in the present invention air is used as the oxidant in the reaction step, such ethane:oxygen molar ratios correspond to ethane:air molar ratios of 0.1:1 to 2.1:1 and 0.1:1 to 1.1:1, respectively.

In a case wherein in the present invention air is used as the oxidant in the reaction step, ethane may be present in the reactor feed in a concentration of at least 5 mole %, more preferably at least 10 mole %, relative to the reactor feed. Further, ethane may be present in the reactor feed in a concentration of at most 70 mole %, more preferably at most 60 mole %, most preferably at most 55 mole %, relative to the reactor feed. Thus, in the present invention, ethane may for example be present in the reactor feed in a concentration in the range of from 5 to 70 mole %, more preferably 10 to 60 mole %, most preferably 10 to 55 mole %, relative to the reactor feed.

In general, the oxygen concentration (wherein the oxygen may or may not originate from air) in the reactor feed should be less than the concentration of oxygen that would form a flammable mixture at either the reactor inlet or the reactor outlet at the prevailing operating conditions.

In a case wherein in the present invention air is used as the oxidant in the reaction step, air may be present in the reactor feed in a concentration of at least 30 mole %, more preferably at least 40 mole %, most preferably at least 45 mole %, relative to the reactor feed. Further, air may be present in the reactor feed in a concentration of at most 95 mole %, more preferably at most 90 mole %, relative to the reactor feed. Thus, in said embodiment, air may for example be present in the reactor feed in a concentration in the range of from 30 to 95 mole %, more preferably 40 to 90 mole %, most preferably 45 to 90 mole %, relative to the reactor feed.

In a case wherein in the present invention oxygen (not air) is used as the oxidant in the reaction step, suitable ethane and oxygen concentrations in the reactor feed can be calculated from the above-mentioned ethane and air concentrations in the reactor feed, taking into account that air comprises 21 mole % of oxygen.

In the above-mentioned reaction step, a reactor feed comprising ethane and oxygen or air is subjected to ethane oxidative dehydrogenation conditions, which as discussed above, may comprise contacting said gas stream with an ethane oxidative dehydrogenation catalyst so that ethane is converted to ethylene. Suitably, the reactor temperature in said reaction step is in the range of from 100 to 600° C. Preferably, said conversion is effected at a reactor temperature in the range of from 200 to 500° C.

In a preferred embodiment, said conversion of ethane to ethylene is effected at a reactor pressure in the range of from 1 to 50 bar, more preferably 3 to 25 bar, even more preferably 5 to 15 bar.

According to the present invention, the above-mentioned ethane oxidative dehydrogenation catalyst may be any ethane oxidative dehydrogenation catalyst. The amount of such catalyst is not essential. Preferably, a catalytically effective amount of the catalyst is used, that is to say an amount sufficient to promote the ethane oxydehydrogenation reaction.

Further, in the present invention, such catalyst may be a mixed metal oxide catalyst containing molybdenum, vanadium, niobium and optionally tellurium as the metals. Thus, in a preferred embodiment of the present invention, the gas stream comprising ethane and oxygen or air is contacted with a mixed metal oxide catalyst containing molybdenum, vanadium, niobium and optionally tellurium, resulting in the gas stream comprising one or more light components, ethane and ethylene.

In the present invention, the above-mentioned mixed metal oxide catalyst containing molybdenum, vanadium, niobium and optionally tellurium may have the following formula:

Mo₁VₐTe_{b}Nb_{c}Oₙ

wherein:

a, b, c and n represent the ratio of the molar amount of the element in question to the molar amount of molybdenum (Mo);

a (for V) is from 0.01 to 1, preferably 0.05 to 0.60, more preferably 0.10 to 0.40, more preferably 0.20 to 0.35, most preferably 0.25 to 0.30;

b (for Te) is 0 or from >0 to 1, preferably 0.01 to 0.40, more preferably 0.05 to 0.30, more preferably 0.05 to 0.20, most preferably 0.09 to 0.15;

c (for Nb) is from >0 to 1, preferably 0.01 to 0.40, more preferably 0.05 to 0.30, more preferably 0.10 to 0.25, most preferably 0.14 to 0.20; and

n (for O) is a number which is determined by the valency and frequency of elements other than oxygen.

In the present invention, the above-mentioned mixed metal oxide catalyst containing molybdenum, vanadium, niobium and optionally tellurium is a solid, heterogeneous catalyst. Inside a reactor, this heterogeneous catalyst makes up a catalyst bed through which the gas stream comprising oxygen or air and ethane is sent.

In general, the gas stream comprising one or more light components, ethane and ethylene resulting from the above-described reaction step also comprises water. Water may easily be removed from said gas stream, for example by cooling down the gas stream from the reaction temperature to a lower temperature, for example to a temperature in the range of from 0 to 50° C., suitably 10 to 40° C. or 10 to 30° C., so that the water condenses and can then be removed from the gas stream. In case any carboxylic acid is formed in the present ethane ODH process, such as acetic acid which is the corresponding carboxylic acid originating from ethane, such carboxylic acid would be separated at the same time together with the water. Therefore, preferably, in an embodiment wherein the gas stream resulting from the above-described reaction step comprises one or more light components, ethane, ethylene, water and optionally carboxylic acid, such water and carboxylic acid are removed from such gas stream in the above-mentioned way, preferably before the below-described first sorption step is carried out.

Such condensing step, as described above, may be followed by a water wash step in order to remove substantially all carboxylic acid, also preferably before the below-described first sorption step is carried out. For example, such water wash may be carried out by contacting the gas stream with water which has an affinity for carboxylic acid.

Further, such condensing step and optional water wash step, as described above, may be followed by a drying step in order to remove substantially all water, also preferably before the below-described first sorption step is carried out. For example, such drying may be carried out by contacting the gas stream with an absorption agent which has a high affinity for water, such as for example triethylene glycol (TEG), for example at a temperature in the range of from 30 to 50° C., suitably about 40° C. Alternatively, such drying may be carried out by contacting the gas stream with molecular sieves (or “mol sieves”), suitably at a relatively low temperature in the range of from 10 to 25° C. Using molecular sieves is preferred in a case where the remaining water content should be as low as possible.

The removal of water and any carboxylic acid before the below-described first sorption step, as described above, is preferred because then advantageously less sorption agent may be used in the latter step since there is less or substantially no water and carboxylic acid to be sorbed by the sorption agent. Further, by removing water and carboxylic acid at this stage, advantageously less or substantially no water and carboxylic acid will interfere with downstream purification of gas streams coming from the below-described sorption steps and/or desorption steps.

Further, in the process of the present invention, the gas stream comprising one or more light components, ethane and ethylene resulting from the above-described reaction step is subjected to a first sorption step. Suitably, in a case wherein in the present invention oxygen (not air) is used as the oxidant in the reaction step, the gas stream that is subjected to the first sorption step comprises 20 to 80 mole % of one or more light components (including e.g. carbon monoxide and unreacted oxygen), more suitably 30 to 70 mole % of one or more light components; 0 to 80 mole % of ethane, more suitably 0 to 70 mole % of ethane; and 0.1 to 65 mole % of ethylene, more suitably 0.5 to 55 mole % of ethylene. Further, suitably, in a case wherein in the present invention air is used as the oxidant in the reaction step, the gas stream that is subjected to the first sorption step comprises 25 to 98 mole % of one or more light components (including e.g. nitrogen, carbon monoxide and unreacted oxygen), more suitably 35 to 90 mole % of one or more light components; 0 to 60 mole % of ethane, more suitably 0 to 50 mole % of ethane; and 0.1 to 20 mole % of ethylene, more suitably 0.5 to 15 mole % of ethylene. All of said relative amounts are based on the total amount of the gas stream.

In the first sorption step of the process of the present invention, the gas stream comprising one or more light components, ethane and ethylene resulting from the reaction step is contacted with a sorption agent which has an affinity for light components which is lower than that for ethane which in turn is lower than that for ethylene, resulting in sorption of ethylene and ethane by the sorption agent and in a gas stream comprising one or more light components and ethane. That is to say, the gas stream resulting from the first sorption step comprises one or more light components and ethane that are not sorbed by the sorption agent. In particular, the amount of ethane in the gas stream resulting from the sorption step is greater than 0% to smaller than 100%, based on the amount of ethane in the gas stream that is subjected to the sorption step. The latter percentage may also be referred to as “ethane rejection” (ethane not being sorbed, but “rejected”). Such “ethane rejection” may be varied by varying the pressure, temperature, nature of the sorption agent and/or configuration of the sorption-desorption system.

In the first desorption step of the present invention, ethane and ethylene that are sorbed by the sorption agent are desorbed, resulting in a gas stream comprising ethane and ethylene. That is to say, the latter gas stream resulting from the first desorption step comprises ethane and ethylene that are desorbed from the sorption agent.

Further, in a second sorption step of the present invention, the gas stream comprising one or more light components and ethane resulting from the first sorption step is contacted with a sorption agent which has an affinity for light components which is lower than that for ethane, resulting in sorption of ethane by the sorption agent and in a gas stream comprising one or more light components. In the second desorption step, ethane that is sorbed by the sorption agent is desorbed, resulting in a gas stream comprising ethane. Optionally, the present invention comprises a recycle step which comprises recycling the gas stream comprising ethane resulting from the second desorption step to the reaction step.

In the first and second sorption steps of the process of the present invention, sorption agents are used. In the present specification, “sorption” means a process in which one substance (the sorption agent) takes up or holds or retains another substance by absorption, adsorption or a combination of both.

Further, a sorption agent used in the first sorption step or the second sorption step of the present invention should have a certain affinity (selectivity). This will be demonstrated hereinbelow with reference to the sorption agent to be used in the first sorption step, but similar principles apply to the sorption agent used in the second sorption step. In said first sorption, the sorption agent has an affinity for light components which is lower than that for ethane which in turn is lower than that for ethylene. This means that under the conditions applied in said sorption step, including pressure and temperature which are further defined hereinbelow, said sorption agent has an affinity for light components which is lower than that for ethane which in turn is lower than that for ethylene. This implies that such sorption agent should be used, that the molar ratio of sorbed ethylene to sorbed light components is greater than 1:1, assuming equal partial pressures for ethylene and light components. Preferably, said ratio is equal to or higher than 1.1:1, more preferably equal to or higher than 5:1. Said ratio may be up to 50:1, or may be up to 40:1, or may be up to 30:1, or may be up to 20:1. For example, said ratio is in the range of from 1.1:1 to 50:1 or from 5:1 to 20:1. Sorption agents suitable to be used in the first sorption step may be selected by comparing the extent of sorption of light components, the extent of sorption of ethane and the extent of sorption of ethylene, under any given temperature and pressure conditions for a variety of known sorption agents, assuming equal partial pressures for light components, ethane and ethylene. Therefore, a wide range of sorption agents may be used since the only criterion for the sorption agent to be used in the first sorption step is that it should have an affinity for light components which is lower than that for ethane which in turn is lower than that for ethylene.

Without any limitation, examples of suitable sorption agents are activated carbons; molecular sieves and zeolites (e.g. zeolite 13X, zeolite 5A, ZSM-5, SAPO-34); mesoporous silicas (e.g. SBA-2, SBA-15); porous silicas (e.g. CMK-3, silicate-1); clay heterostructures; Engelhard Titanosilicates (ETS; e.g. ETS-4, ETS-10); porous coordination polymers (PCPs); cation impregnated porous adsorbents and zeolites (e.g. AgA); metal-organic frameworks (MOFs); Zeolitic Imidazolate Framework (ZIFs); and Carbon Organic Frameworks (COFs). Suitable sorption agents are for example disclosed in US20150065767 and US20140249339.

In the first and second sorption steps of the present invention, the same or a different sorption agent may be used. A different sorption agent (having a different selectivity) may be used in a case where the pressure in these sorption steps is the same, for example in a case where the gas stream resulting from the first sorption step is sent to the second sorption step without any further compression, as described below. Alternatively or additionally, in such case, the temperature and/or configuration of the sorption-desorption system may be varied. In a case where the same sorption agent is used, the pressure, temperature and/or configuration of the sorption-desorption system may be varied in order to achieve the different selective sorption.

The pressure in the first and second sorption steps of the process of the present invention may vary within wide ranges. Preferably, said pressure is higher than atmospheric pressure and at most 30 bar, more preferably at most 15 bar. More preferably, said pressure is of from 5 to 30 bar, more preferably 5 to 15 bar, most preferably 7 to 13 bar. In the reaction step of the process of the present invention, oxygen or air may be fed at a pressure in the range of from 5 to 30 bar, preferably 5 to 15 bar, more preferably 7 to 13 bar. This implies advantageously that the gas stream comprising one or more light components, ethane and ethylene resulting from the reaction step generally need not be compressed before subjecting it to the first sorption step. Thus, preferably, the pressure at which oxygen or air is fed in the reaction step is the same as the pressure in the first sorption step.

The temperature in the first and second sorption steps of the process of the present invention may also vary within wide ranges. Preferably, said temperature is in the range of from 0 to 110° C., more preferably 10 to 90° C., most preferably 25 to 80° C. Advantageously, in the present invention, said sorption steps may be carried out at a non-cryogenic temperature (e.g. of from 0 to 110° C. as mentioned above).

Preferably, in the first and second desorption steps of the process of the present invention, desorption is effected by reducing the pressure. That is to say, the pressure in a certain desorption step (for example first desorption step) is lower than the pressure in the sorption step that directly precedes said desorption step (for example first sorption step). This is usually referred to as “Pressure Swing Adsorption” (PSA). In case desorption in the first and second desorption steps is effected by reducing the pressure, the pressure in those sorption steps is preferably in the range of from 5 to 30 bar, more preferably 5 to 15 bar, more preferably 7 to 13 bar.

Advantageously, in the first and second sorption steps of the present invention, a relatively low pressure may be applied (e.g. of from 5 to 15 bar as mentioned above). Such low pressure advantageously results in that relatively less compression of the gas stream may be needed. It is especially advantageous that the pressure that may be needed in said sorption steps may be the same as the pressure in the (ODH) reaction step. In the latter case, there would be no need at all for any compression of said gas stream in order to carry out the sorption steps.

A further advantage of the present invention is that two sorption steps are carried out. In the present invention, this implies advantageously that the gas stream resulting from the first sorption step may be sent to the second sorption step without any further compression. Furthermore, advantageously, in the first sorption step of the process of the present invention it is allowed that a relatively large portion of the (unconverted) ethane is not sorbed and slips into the gas stream comprising one or more light components and ethane resulting from the first sorption step. This means that the separation of ethane in the first sorption step need not be sharp, since part of the ethane is sorbed and the remaining part is not sorbed, which is advantageous in that a sharper separation between light components and ethylene is achieved. That not-sorbed portion of ethane is then separated from light components in the second sorption step of the process of the present invention, at the same pressure as discussed before (no further compression), so that the overall hydrocarbon efficiency is also advantageously increased. Therefore, this also advantageously implies that the energy demand on a splitter, and associated capital expenditure, that would be needed to separate sorbed ethane from sorbed ethylene (after desorption), are reduced since only a portion of the ethane was sorbed previously. Furthermore, said second sorption step advantageously also takes away the need to separate light components using cryogenic distillation under high pressure, including a complicated cascade refrigeration system, wherein light components would first be separated from the ethane and ethylene.

Further, in the embodiment wherein desorption in the first and second desorption steps is effected by reducing the pressure, the pressure in the desorption steps is preferably in the range of from 0.1 to 3 bar, more preferably 0.5 to 2 bar.

The temperature in the first and second desorption steps of the process of the present invention may also vary within wide ranges. Preferably, said temperature is in the range of from 0 to 110° C., more preferably 10 to 90° C., most preferably 25 to 80° C. Advantageously, in the present invention, said desorptions step may be carried out at a non-cryogenic temperature (e.g. of from 0 to 110° C. as mentioned above).

Advantageously, the sorption and desorption steps of the process of the present invention make it possible to first efficiently separate one or more light components and ethane from a gas stream comprising one or more light components, ethane and ethylene resulting from the (ODH) reaction step, and then efficiently separate light components from a gas stream comprising one or more light components and ethane resulting from the first sorption step, at a relatively low pressure (e.g. at most 15 bar as mentioned above) and at a non-cryogenic temperature (e.g. of from 0 to 110° C. as mentioned above).

Preferably, the gas stream comprising one or more light components, ethane and ethylene that is subjected to the first sorption step of the process of the present invention comprises substantially no water. As described above, preferably, any water is removed from said gas stream before the first sorption step is carried out. It is also preferred that said gas stream comprising one or more light components, ethane and ethylene comprises substantially no hydrogen sulfide.

Preferably, the present process additionally comprises a distillation step which comprises distilling the gas stream comprising ethane and ethylene resulting from the first desorption step, resulting in a top stream comprising ethylene and a bottom stream comprising ethane; and optionally a recycle step which comprises recycling the bottom stream comprising ethane resulting from the distillation step to the reaction step.

As is demonstrated in the present Examples, it has surprisingly appeared that advantageously the energy demand, especially the demand for compression and refrigeration energy, is significantly lower as compared to a process wherein a sorption and desorption method, comprising multiple sorption and desorption steps, is not applied after the ODH reaction step, irrespective of whether in the ODH reaction step of such comparative case only oxygen (no nitrogen) or air is used. Thus, the present process is a process that enables the oxidative dehydrogenation of ethane and subsequent separation of the product stream comprising one or more light components, ethane and ethylene, to recover unconverted ethane and ethylene, in a way that is technically feasible, efficient and affordable since the energy demand is surprisingly lower as compared to the comparative process.

Further, in an embodiment of the process of the present invention, the gas stream comprising one or more light components, ethane and ethylene that is subjected to the first sorption step of the process of the present invention additionally comprises components other than said one or more light components (as defined above), ethane and ethylene, such as carbon dioxide. Therefore, in the present invention, the reaction step may result in a gas stream comprising one or more light components, ethane, ethylene and carbon dioxide.

In the above-mentioned embodiment of the process of the present invention, wherein the reaction step results in a gas stream comprising one or more light components, ethane, ethylene and carbon dioxide,

the first sorption step comprises contacting the gas stream comprising one or more light components, ethane, ethylene and carbon dioxide resulting from the reaction step with a sorption agent which has an affinity for light components which is lower than that for ethane which in turn is lower than that for ethylene and carbon dioxide, resulting in sorption of ethylene, ethane and carbon dioxide by the sorption agent and in a gas stream comprising one or more light components and ethane;

the first desorption step comprises desorbing sorbed ethylene, ethane and carbon dioxide resulting in a gas stream comprising ethylene, ethane and carbon dioxide;

the second sorption step comprises contacting the gas stream comprising one or more light components and ethane resulting from the first sorption step with a sorption agent which has an affinity for light components which is lower than that for ethane, resulting in sorption of ethane by the sorption agent and in a gas stream comprising one or more light components; and

the second desorption step comprises desorbing ethane sorbed in the second sorption step resulting in a gas stream comprising ethane.

The sorption agents, pressures, temperatures, sorption-desorption method (e.g. PSA) and configuration of the sorption-desorption system as discussed above also apply to the above-mentioned embodiment of the process of the present invention, wherein the gas stream that is subjected to the first sorption step (gas stream resulting from the reaction step) comprises one or more light components, ethane, ethylene and carbon dioxide.

Further, preferably, in the above-mentioned embodiment, the process of the present invention additionally comprises a carbon dioxide removal step which comprises removing carbon dioxide from the gas stream comprising carbon dioxide, ethylene and ethane resulting from the first desorption step, resulting in a gas stream comprising ethylene and ethane. In said carbon dioxide removal step, carbon dioxide may be removed by any known method, such as treatment with an amine and then with a caustic agent, such as an aqueous monoethanolamine (MEA) absorption system and aqueous NaOH, respectively, as already mentioned above in the introduction of this specification. In a case where said carbon dioxide removal step involves the use of water, said step normally also involves the removal of that water, suitably followed by a drying step. Such drying step may be carried out in order to remove substantially all water and may be carried out in one of the ways as exemplified above in relation to the optional drying step after a condensing step.

The above-described embodiment of the process of the present invention, comprising the first and second sorption and desorption steps followed by the carbon dioxide removal step, may additionally comprise a distillation step wherein the gas stream comprising ethylene and ethane resulting from the carbon dioxide removal step as carried out after the first desorption step is distilled. Preferably, said distillation step comprises distilling the gas stream comprising ethylene and ethane resulting from the carbon dioxide removal step, resulting in a top stream comprising ethylene and a bottom stream comprising ethane. Optionally, said bottom stream comprising ethane is recycled to the reaction step.

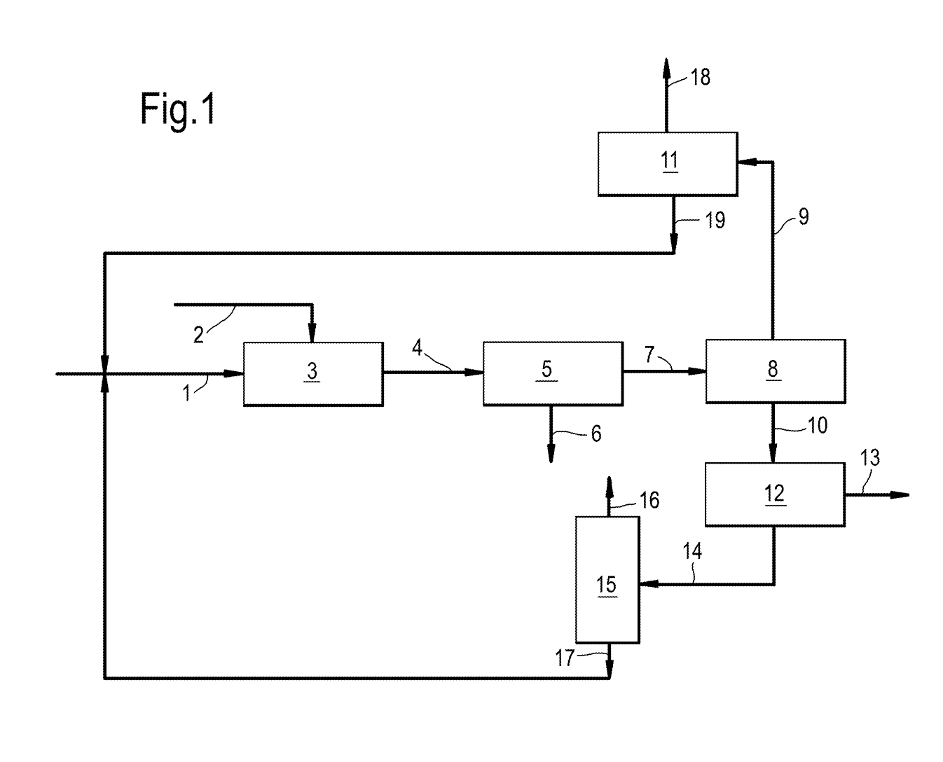

An example of said embodiment of the process of the present invention, wherein the gas stream that is subjected to the first sorption step additionally comprises components other than one or more light components, ethane and ethylene, namely carbon dioxide, is schematically shown in FIG. 1.

In said FIG. 1, a gas stream 1 comprising ethane and an air stream 2 are fed to an ethane oxidative dehydrogenation (ODH) reactor 3 containing an ODH catalyst and operating under ODH conditions. Product stream 4 originating from ODH reactor 3 comprises water, ethane, ethylene, hydrogen, nitrogen, carbon monoxide and carbon dioxide. Said stream 4 is fed to condensation vessel 5 where water is removed via stream 6. Gas stream 7 comprising ethane, ethylene, hydrogen, nitrogen, carbon monoxide and carbon dioxide originating from condensation vessel 5 is fed to first sorption and desorption unit 8. Optionally, before gas stream 7 is fed to first sorption and desorption unit 8, it is sent to a drying unit (not shown in FIG. 1) in order to remove substantially all water.

Sorption and desorption unit 8 comprises a sorption agent which has an affinity for hydrogen, nitrogen and carbon monoxide which is lower than that for ethane which in turn is lower than that for carbon dioxide and ethylene. The pressure of gas stream 7 may be of from 5 to 15 bar. Carbon dioxide, ethane and ethylene are sorbed by the sorption agent. Further, a gas stream 9 comprising nitrogen, hydrogen, carbon monoxide and ethane leaves sorption and desorption unit 8, which nitrogen, hydrogen, carbon monoxide and ethane are not sorbed by the sorption agent in sorption and desorption unit 8. Gas stream 9 is sent to sorption and desorption unit 11.

After some time, the feed of gas stream 7 to sorption and desorption unit 8 is stopped and the pressure in said unit is reduced. For example, the pressure in sorption and desorption unit 8 may be reduced to a pressure in the range of from 0.1 to 3 bar in a case wherein during the sorption step the pressure is in the range of from 5 to 15 bar, as exemplified above. Through such pressure reduction carbon dioxide, ethane and ethylene that are sorbed by the sorption agent become desorbed. A gas stream 10 comprising carbon dioxide, ethane and ethylene, that are desorbed from the sorption agent, leaves sorption and desorption unit 8 and is sent to carbon dioxide removal unit 12.

Once the desorption is completed, the feed of gas stream 7 to sorption and desorption unit 8 is resumed and the above procedure is repeated.

Sorption and desorption unit 11 comprises a sorption agent which has an affinity for hydrogen, nitrogen and carbon monoxide which is lower than that for ethane. The pressure of gas stream 9 may be of from 5 to 15 bar. Ethane is sorbed by the sorption agent. Further, a gas stream 18 comprising nitrogen, hydrogen and carbon monoxide leaves sorption and desorption unit 11, which nitrogen, hydrogen and carbon monoxide are not sorbed by the sorption agent in sorption and desorption unit 11.

After some time, the feed of gas stream 9 to sorption and desorption unit 11 is stopped and the pressure in said unit is reduced. For example, the pressure in sorption and desorption unit 11 may be reduced to a pressure in the range of from 0.1 to 3 bar in a case wherein during the sorption step the pressure is in the range of from 5 to 15 bar, as exemplified above. Through such pressure reduction ethane that is sorbed by the sorption agent becomes desorbed. A gas stream 19 comprising ethane that is desorbed from the sorption agent, leaves sorption and desorption unit 11 and is recycled to ODH reactor 3.

Once the desorption is completed, the feed of gas stream 9 to sorption and desorption unit 11 is resumed and the above procedure is repeated.

In carbon dioxide removal unit 12, carbon dioxide is removed, via stream 13, from gas stream 10 comprising carbon dioxide, ethane and ethylene, in a way as exemplified above, that is to say involving the use of water and the removal of that water. A gas stream 14 comprising ethane and ethylene leaves carbon dioxide removal unit 12. Before gas stream 14 is sent to distillation column 15, it is sent to a drying unit (not shown in FIG. 1) in order to remove substantially all water.

In distillation column 15, gas stream 14 comprising ethane and ethylene is distilled such that separation between on the one hand ethylene and on the other hand ethane is effected. That is, a top stream 16 comprising ethylene and a bottom stream 17 comprising ethane leave distillation column 15. Said bottom stream 17 is advantageously recycled to ODH reactor 3, for further conversion of the recovered ethane.

If in the setup of FIG. 1, a gas stream 1 comprising ethane of sufficiently high pressure (for example in the range of from 5 to 15 bar) is fed to ODH reactor 3, gas compressors would advantageously only be needed in line 2 (compression of air); in line 10 (compression of gas stream 10 leaving sorption and desorption unit 8, after desorption, and entering carbon dioxide removal unit 12); and in line 19 (compression of gas stream 19 leaving sorption and desorption unit 11, after desorption, and entering ODH reactor 3).

The invention is further illustrated by the following Examples.

### EXAMPLES A AND C AND COMPARATIVE EXAMPLES B AND D

In Example A exemplifying the present invention and in Comparative Example B, a gas stream comprising ethane having a temperature of 40° C. and a pressure of 10 bar is fed to an ethane oxidative dehydrogenation (ODH) reactor. In addition, a gas stream comprising oxygen, and having a temperature of 40° C. and a pressure of 10 bar, is fed to the ODH reactor.

Said gas stream comprising oxygen is produced in the following way. A gas stream comprising air of ambient temperature and pressure is fed to an air separation unit (ASU). The ASU is operated such that the following 2 streams leave the ASU: 1) a gas stream comprising nitrogen having a temperature of 40° C. and a pressure of 20 bar (which nitrogen can be subsequently stored); and 2) a gas stream comprising oxygen (purity of 99.5 mole %; 0.5 mole % of nitrogen) having a temperature of 40° C. and a pressure of 10 bar.

Said gas stream comprising ethane and said gas stream comprising oxygen form a combined gas stream comprising ethane and oxygen inside the ODH reactor, which combined gas stream comprises 53 mole % of ethane and 47 mole % of oxygen (ethane:oxygen molar ratio=1.1). The ODH reactor contains an ethane oxidative dehydrogenation (ODH) catalyst and is operated under ODH conditions, including a temperature in the range of from 200 to 500° C. and a pressure of 10 bar. The conversion of ethane is 80%, the selectivity to ethylene is 80% and the selectivity to carbon monoxide and carbon dioxide is 20%.

In Example A and in Comparative Example B, a product stream comprising 8.4 mole % of ethane, 4.2 mole % of oxygen, 26.9 mole % of ethylene, 6.7 mole % of carbon monoxide, 6.7 mole % of carbon dioxide and 47.1 mole % of water leaves the ODH reactor. Said product stream is cooled to a temperature of 40° C., thereby condensing out the water which is then separated. Then the carbon dioxide is removed from the resulting product stream in a carbon dioxide removal step. Any remaining water in the resulting product stream is then removed in a drying unit. After said water and carbon dioxide removal, said product stream is a gas stream comprising 18.2 mole % of ethane, 9.1 mole % of oxygen, 58.2 mole % of ethylene and 14.5 mole % of carbon monoxide.

In Example A, the ODH reactor product stream from which water and carbon dioxide are removed is fed to a first sorption and desorption unit, which comprises a sorption agent which has an affinity for light components (including oxygen and carbon monoxide) which is lower than that for ethane which in turn is lower than that for ethylene. All of the ethylene and 50% of the ethane are sorbed by the sorption agent (ethylene rejection=0%; ethane rejection=50%). A gas stream comprising oxygen, carbon monoxide and ethane, and having a temperature of 40° C. and a pressure of 10 bar, leaves the first sorption and desorption unit, which oxygen, carbon monoxide and ethane are not sorbed by the sorption agent in the first sorption and desorption unit.

In Example A, the gas stream comprising oxygen, carbon monoxide and ethane which leaves the first sorption and desorption unit, is fed at the same pressure (10 bar) to a second sorption and desorption unit, which comprises a sorption agent which has an affinity for light components (including oxygen and carbon monoxide) which is lower than that for ethane. All of the ethane is sorbed by the sorption agent (ethane rejection=0%; oxygen and carbon monoxide rejection=100%). A gas stream comprising oxygen and carbon monoxide, and having a temperature of 40° C. and a pressure of 10 bar, leaves the second sorption and desorption unit, which oxygen and carbon monoxide are not sorbed by the sorption agent in the second sorption and desorption unit.

After some time, in Example A, the feed of the gas stream to the first sorption and desorption unit is stopped and the pressure in said unit is reduced from 10 bar to 1 bar, thereby inducing a first desorption step. The sorbed components (ethylene and ethane) subsequently become desorbed from the sorption agent and leave the first sorption and desorption unit at a temperature of 40° C. and a pressure of 1 bar. In Example A, the latter gas stream is advantageously enriched in ethylene as compared to the gas stream that is fed to the first sorption and desorption unit: the gas stream leaving the first sorption and desorption unit upon desorption comprises all of the ethylene and 50% of the ethane from the product stream.

After some time, in Example A, the feed of the gas stream to the second sorption and desorption unit is stopped and the pressure in said unit is reduced from 10 bar to 1 bar, thereby inducing a second desorption step. The sorbed components (ethane) subsequently become desorbed from the sorption agent and leave the second sorption and desorption unit at a temperature of 40° C. and a pressure of 1 bar. The gas stream leaving the second sorption and desorption unit upon desorption comprises 50% of the ethane from the product stream. The latter gas stream comprising ethane is compressed to 10 bar by a compressor comprising 3 compression stages, after which the gas stream may be recycled to the ODH reactor.

In Example A, the gas stream comprising ethylene and ethane which leaves the first sorption and desorption unit upon desorption is compressed to 16.5 bar by a compressor comprising 3 compression stages and then cooled to a temperature of −34° C. in two parallel heat exchangers utilizing the low temperature of the top and bottom streams coming from below-described distillation column A. Then said stream is fed to a distillation column having 99 theoretical stages, hereinafter referred to as distillation column A, and distilled, resulting in a top stream comprising ethylene and having a temperature of −38° C. and a pressure of 15.5 bar and in a bottom stream comprising ethane and having a temperature of −16° C. and a pressure of 16 bar. Said top and bottom streams are used to cool the feed streams in order to minimize condenser duty in distillation column A, which is provided by a propane refrigeration cycle.

In Comparative Example B, the ODH reactor product stream from which water and carbon dioxide are removed is compressed to 18 bar by a compressor comprising 1 compression stage and then cooled to a temperature of −44° C. in three parallel heat exchangers utilizing the low temperature of the top stream coming from below-described distillation column B and the top and bottom streams coming from below-described distillation column C Then said stream is fed to a distillation column having 6 theoretical stages, hereinafter referred to as distillation column B, and distilled, resulting in a top stream comprising oxygen and carbon monoxide and having a temperature of −145° C. and a pressure of 16 bar and in a bottom stream comprising ethane and ethylene and having a temperature of −33° C. and a pressure of 16 bar. Said top stream is used to cool the feed streams in order to minimize condenser duty in distillation column B, which is provided by a cascaded methane-ethylene-propane refrigeration cycle.

In Comparative Example B, the bottom stream comprising ethane and ethylene coming from distillation column B is fed to a distillation column having 99 theoretical stages, hereinafter referred to as distillation column C, and distilled, resulting in a top stream comprising ethylene and having a temperature of −38° C. and a pressure of 15.5 bar and in a bottom stream comprising ethane and having a temperature of −16° C. and a pressure of 16 bar. Said top and bottom streams are used to cool the feed streams in order to minimize condenser duty in distillation column B

Example C, also exemplifying the present invention (just like Example A), is performed in the same way as Example A, with the exception that a gas stream comprising air (that is to say, nitrogen and oxygen instead of oxygen alone), and having a temperature of 40° C. and a pressure of 10 bar, is fed to the ODH reactor. A product stream comprising 3.5 mole % of ethane, 1.7 mole % of oxygen, 58.7 mole % of nitrogen, 11.1 mole % of ethylene, 2.8 mole % of carbon monoxide, 2.8 mole % of carbon dioxide and 19.4 mole % of water leaves the ODH reactor. After the water and carbon dioxide removal, said product stream is a gas stream comprising 4.5 mole % of ethane, 2.2 mole % of oxygen, 75.5 mole % of nitrogen, 14.3 mole % of ethylene and 3.6 mole % of carbon monoxide.

Comparative Example D is performed in the same way as Example C, with the exception that the ODH reactor product stream from which water and carbon dioxide are removed is compressed to 18 bar by a compressor comprising 1 compression stage and then cooled to a temperature of −101° C. in four parallel heat exchangers utilizing the low temperature of the top stream coming from below-described distillation column D and the top and bottom streams coming from distillation column C Then said stream is fed to a distillation column having 6 theoretical stages, hereinafter referred to as distillation column D, and distilled, resulting in a top stream comprising nitrogen, oxygen and carbon monoxide and having a temperature of −159° C. and a pressure of 17.5 bar and in a bottom stream comprising ethane and ethylene and having a temperature of −30° C. and a pressure of 18 bar. Said top stream is used to cool the feed streams in order to minimize condenser duty in distillation column D, which is provided by a cascaded methane-ethylene-propane refrigeration cycle.

In Comparative Example D, the bottom stream comprising ethane and ethylene coming from distillation column D is separated in the same way using distillation column C as described above for Comparative Example B.

In Table 1 below, the reflux ratios and the distillate-to-feed ratios needed to achieve the above separations in distillation columns A, B, C and D in Examples A and C and Comparative Examples B and D are mentioned. By said “reflux ratio”, reference is made to the molar ratio of the molar flow rate of the “reflux stream”, which is that part of the stream that leaves the condenser at the top of the distillation column which is sent back to that column, divided by the molar flow rate of the “distillate”, which is that part of the stream that leaves the condenser at the top of the distillation column which is not sent back to that column. By said “distillate-to-feed ratio”, reference is made to the molar ratio of the molar flow rate of said “distillate” divided by the molar flow rate of the feed stream that is fed to that column (the “feed”).

TABLE 1

| | | | Distillate-to-feed |
|---|---|---|---|
| Example(s) | Distillation column | Reflux ratio | ratio |
| A + C | A | 2.2 | 0.86 |
| B* | B | 4.2 | 0.25 |
| B* + D* | C | 2.4 | 0.75 |
| D* | D | 0.7 | 0.81 |

*= comparative

In all of the (Comparative) Examples, ethane containing streams separated in the distillation columns may be recycled to the ODH reactor at 10 bar. The temperature reduction by reducing the pressure of such recycle ethane containing streams to 10 bar, as well as the temperature reduction by reducing the pressure of nitrogen, carbon monoxide and oxygen containing top (vent) streams to atmospheric pressure, are utilized to cool the feed streams to the distillation columns and in this way the condenser duty provided by refrigeration is reduced.

In Table 2 below, the compression and refrigeration energy needed to convert ethane into ethylene and to separately recover ethane and ethylene from the product stream is included for all of Examples A and C and Comparative Examples B and D. Said energy is expressed as kilowatt hour (“kWh”; 1 kWh=3.6 megajoules) per kilogram (kg) of ethylene.

TABLE 2

| | | kWh/kg of |
|---|---|---|
| Ex. | Configuration | ethylene |
| A | O₂ [no N₂] | 1.53 |
| | 1^{st} PSA [100% CO + O₂ rejection + 50% ethane | |
| | rejection] | |
| | 1 distillation separating desorbed ethylene and ethane | |
| | 2^{nd} PSA [100% CO + O₂ rejection] | |
| B* | O₂ [no N₂] + distillation only [no PSA] | 1.80 |
| C | air [O₂ + N₂] | 0.82 |
| | 1^{st} PSA [100% CO + N₂ + O₂ rejection + 50% ethane | |
| | rejection] | |
| | 1 distillation separating desorbed ethylene and ethane | |
| | 2^{nd} PSA [100% CO + N₂ + O₂ rejection] | |
| D* | air [O₂ + N₂] + distillation only [no PSA] | 1.24 |

*= comparative

From Table 2 above, it surprisingly appears that the energy needed to convert ethane into ethylene and to separately recover ethane and ethylene from the product stream is advantageously lowest in case the process of the present invention is carried out. That is, in all of Examples A and C, which exemplify the process of the present invention wherein in the ODH reaction step oxygen or air is used and in the subsequent product separation steps multiple sorption and desorption methods (in said Examples: PSA methods) are applied, said energy is advantageously lower than the energy needed to effect the same in those cases wherein sorption and desorption methods are not applied after the ODH reaction step, but only distillation steps are performed (as in Comparative Examples B and D), both when only oxygen (no nitrogen) is used in the ODH reaction step (Example A and Comparative Example B) and when air is used in the ODH reaction step (Example C and Comparative Example D). In the latter case (air), the advantageous different energy effect obtained with the process of the present invention is surprisingly largest.

Thus, surprisingly, this advantageous different energy effect obtained with the process of the present invention, as compared to the processes wherein only distillation steps are performed, is even obtained in cases where the first sorption and desorption steps are followed by 1 distillation step (Examples A and C) to recover the ethane and ethylene.

What is claimed is:

1. A process for the oxidative dehydrogenation of ethane to ethylene, comprising
a reaction step which comprises subjecting a gas stream comprising ethane and oxygen to ethane oxidative dehydrogenation conditions resulting in a gas stream comprising one or more light components, ethane and ethylene;
a first sorption step which comprises contacting the gas stream comprising one or more light components, ethane and ethylene resulting from the reaction step with a sorption agent which has an affinity for light components which is lower than that for ethane which in turn is lower than that for ethylene, resulting in sorption of ethylene and ethane by the sorption agent and in a gas stream comprising one or more light components and ethane;
a first desorption step which comprises desorbing sorbed ethylene and ethane resulting in a gas stream comprising ethylene and ethane;
a second sorption step which comprises contacting the gas stream comprising one or more light components and ethane resulting from the first sorption step with a sorption agent which has an affinity for light components which is lower than that for ethane, resulting in sorption of ethane by the sorption agent and in a gas stream comprising one or more light components;
a second desorption step which comprises desorbing ethane sorbed in the second sorption step resulting in a gas stream comprising ethane; and
optionally a recycle step which comprises recycling the gas stream comprising ethane resulting from the second desorption step to the reaction step.

2. The process according to claim 1, comprising
a reaction step which comprises subjecting a gas stream comprising ethane and air to ethane oxidative dehydrogenation conditions resulting in a gas stream comprising nitrogen, ethane and ethylene;
a first sorption step which comprises contacting the gas stream comprising nitrogen, ethane and ethylene resulting from the reaction step with a sorption agent which has an affinity for nitrogen which is lower than that for ethane which in turn is lower than that for ethylene, resulting in sorption of ethane and ethylene by the sorption agent and in a gas stream comprising nitrogen and ethane;
a first desorption step which comprises desorbing ethane and ethylene sorbed in the first sorption step resulting in a gas stream comprising ethane and ethylene;
a second sorption step which comprises contacting the gas stream comprising nitrogen and ethane resulting from the first sorption step with a sorption agent which has an affinity for nitrogen which is lower than that for ethane, resulting in sorption of ethane by the sorption agent and in a gas stream comprising nitrogen;
a second desorption step which comprises desorbing ethane sorbed in the second sorption step resulting in a gas stream comprising ethane; and
a recycle step which comprises recycling the gas stream comprising ethane resulting from the second desorption step to the reaction step.

3. The process according to claim 1, additionally comprising
a distillation step which comprises distilling the gas stream comprising ethane and ethylene resulting from the first desorption step, resulting in a top stream comprising ethylene and a bottom stream comprising ethane; and
a recycle step which comprises recycling the bottom stream comprising ethane resulting from the distillation step to the reaction step.

4. The process according to claim 1, wherein desorption in the first and second desorption steps is effected by reducing the pressure.

5. The process according to claim 4, wherein the pressure in the first and second sorption step is in the range of from 5 to 30 bar, and the pressure in the first and second desorption step is in the range of from 0.1 to 3 bar.

6. The process according to claim 5, wherein in the reaction step oxygen or air is fed at a pressure in the range of from 5 to 15 bar.
